(19) United States
(12) Patent Application Publication
(10) Pub. No.: US 2025/0221653 A1
(43) Pub. Date: Jul. 10, 2025

(54) CARDIAC EVENT ASSESSMENT

(71) Applicant: Cardiac Pacemakers, Inc., St Paul, MN (US)

(72) Inventors: Deepa Mahajan, North Oaks, MN (US); Sai Sanjay Balaji, Minneapolis, MN (US); Ramesh Wariar, Blaine, MN (US); Jonathan H Kelly

(21) Appl. No.: 19/008,058

(22) Filed: Jan. 2, 2025

Related U.S. Application Data
(60) Provisional application No. 63/617,503, filed on Jan. 4, 2024.

(51) Int. Cl. A61B 5/352; A61B 5/00; A61B 5/339; G16H 50/20

(52) U.S. Cl. CPC A61B 5/352; A61B 5/339; A61B 5/4842; A61B 5/746; A61B 5/748; G16H 50/20; A61B 5/7435

(57) ABSTRACT

Systems, methods, and devices involve receiving a package of data that includes an electrocardiogram (ECG) waveform associated with a potential cardiac event, processing the ECG waveform to extract interval data associated with the potential cardiac event, inputting the ECG waveform and the interval data into a trained machine learning model, and determining, by the trained machine learning model, that the potential cardiac event comprises a normal rhythm.

### CROSS REFERENCE TO RELATED APPLICATION

This application claims priority to Provisional Application No. 63/617,503, filed Jan. 4, 2024, which is herein incorporated by reference in its entirety.

### TECHNICAL FIELD

The present disclosure relates generally to approaches for assisting with efficient evaluation of health events detected by medical devices.

### BACKGROUND

Medical devices that allow physicians to monitor cardiac activity are becoming increasingly common in diagnosing and treating medical conditions in patients. Cardiac monitoring can be used, for example, to identify abnormal cardiac rhythms, so that critical alerts can be provided to patients, physicians, or other care providers and so that patients can be treated as needed.

### SUMMARY

In Example 1, a method includes receiving a package of data comprising an electrocardiogram (ECG) waveform associated with a potential cardiac event, processing the ECG waveform to extract interval data associated with the potential cardiac event, inputting the ECG waveform and the interval data into a trained machine learning model, and determining, by the trained machine learning model, that the potential cardiac event comprises a normal rhythm.

In Example 2, the method of Example 1, further including generating an alert in response to the determining that the potential cardiac event contains the normal cardiac rhythm.

In Example 3, the method of Example 2, further including displaying the alert and the ECG waveform on a display of a mobile computing device, wherein the mobile computing device operates the trained machine learning model.

In Example 4, the method of any of the preceding Examples, wherein the interval data comprises data relating to peaks of R waves.

In Example 5, the method of any of the preceding claims, further including processing the ECG waveform to generate non-linear features and inputting the non-linear features into the trained machine learning model.

In Example 6, the method of any of the preceding Examples, further including preventing the potential cardiac event from being forwarded to a physician.

In Example 7, the method of any of the preceding Examples, wherein the trained machine learning model comprises classification model.

In Example 8, the method of any of the preceding Examples, wherein the trained machine learning model comprises an ensemble of boosted trees.

In Example 9, the method of any of the preceding Examples, wherein the normal rhythm is a normal sinus rhythm.

In Example 10, the method of any of the preceding Examples, further including receiving input from a patient indicating that the patient is experiencing the potential cardiac event.

In Example 11, the method of Example 10, further including transmitting a command to a medical device to record the ECG waveform, in response to the input from the patient.

In Example 12, the method of Example 11, further including recording the ECG waveform, by the medical device, in response to the medical device receiving the command.

In Example 13, a computer program product comprising instructions to cause one or more processors to carry out the method of Examples 1-11.

In Example 14, memory having stored thereon the computer program product of Example 13.

In Example 15, a mobile computing device comprising the memory of Example 14.

In Example 16, a system including a mobile computing device with memory and one or more processors. The memory stores instructions that, when executed, cause the mobile computing device to: process ECG waveform associated with a potential cardiac event to extract interval data associated with the potential cardiac event, input the ECG waveform and the interval data into a trained machine learning model, and determine, by the trained machine learning model, that the potential cardiac event comprises a normal rhythm.

In Example 17, the system of Example 16, wherein the instructions, when executed, further cause the mobile computing device to: generate an alert in response to the determining that the potential cardiac event contains the normal cardiac rhythm.

In Example 18, the system of Example 17, wherein the instructions, when executed, further cause the mobile computing device to: display the alert and the ECG waveform on a display of the mobile computing device.

In Example 19, the system of Example 16, wherein the interval data comprises data relating to peaks of R waves.

In Example 20, the system of Example 19, wherein the data relating to peaks of R waves includes a time interval between successive R waves.

In Example 21, the system of Example 16, wherein the instructions, when executed, further cause the mobile computing device to: process the ECG waveform to generate non-linear features and input the non-linear features into the trained machine learning model.

In Example 22, the system of Example 16, wherein the instructions, when executed, further cause the mobile computing device to: prevent the potential cardiac event from being forwarded to a physician.

In Example 23, the system of Example 16, wherein the trained machine learning model comprises classification model.

In Example 24, the system of Example 16, wherein the trained machine learning model comprises an ensemble of boosted trees.

In Example 25, the system of Example 16, wherein the normal rhythm is a normal sinus rhythm.

In Example 26, the system of Example 16, wherein the instructions, when executed, further cause the mobile computing device to: transmit a command to a medical device to record the ECG waveform, in response to input from the patient.

In Example 27, the system of Example 26, wherein the mobile computing device includes a display configured to display a user interface, wherein the input is a selection of an icon on the user interface.

In Example 28, the system of Example 26, further including the medical device communicatively coupled to the mobile computing device, wherein the medical device is programmed to record the ECG waveform in response to the medical device receiving the command.

In Example 29, the system of Example 16, wherein the mobile computing device is programmed to determine, via the trained machine learning model, that the potential cardiac event is only either an abnormal cardiac event or a normal cardiac event.

In Example 30, a method includes receiving a package of data comprising an ECG waveform associated with a potential cardiac event, processing the ECG waveform to extract interval data associated with the potential cardiac event, inputting the ECG waveform and the interval data into a trained machine learning model, and determining, by the trained machine learning model, that the potential cardiac event is either a normal cardiac event or an abnormal cardiac event.

In Example 31, the method of Example 30, further including generating an alert in response to the determining that the potential cardiac event is the normal cardiac event and displaying the alert and the ECG waveform on a display of a mobile computing device.

In Example 32, the method of Example 30, wherein the interval data comprises data relating to peaks of R waves.

In Example 33, the method of Example 30, further including preventing the potential cardiac event from being forwarded to a physician.

In Example 34, the method of Example 30, further including receiving input from a patient indicating that the patient is experiencing the potential cardiac event and transmitting a command to a medical device to record the ECG waveform, in response to the input from the patient.

In Example 35, the method of Example 34, further including recording the ECG waveform, by the medical device, in response to the medical device receiving the command.

In Example 36, the system or methods of any of the preceding Examples, further including determining that the potential cardiac event comprises a normal rhythm based, at least in part, on sensor data indicating patient activity.

In Example 37, the system or methods of any of the preceding Examples, further including determining that the potential cardiac event comprises a normal rhythm based, at least in part, on additional sensed cardiac activity such as heart sounds or a second ECG waveform.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration of a system that includes an implantable medical device, in accordance with certain instances of the present disclosure.

FIG. 2 is a block diagram illustrating a method for assessing potential cardiac events, in accordance with certain instances of the present disclosure.

FIG. 3 is schematic of a receiver such as a mobile electronic device with a display, in accordance with certain instances of the present disclosure.

FIG. 4 is a block diagram depicting an illustrative computing device, in accordance with instances of the disclosure.

While the disclosure is amenable to various modifications and alternative forms, specific instances have been shown by way of example in the drawings and are described in detail below. The intention, however, is not to limit the disclosure to the particular instances described. On the contrary, the disclosure is intended to cover all modifications, equivalents, and alternatives falling within the scope of the disclosure as defined by the appended claims.

### DETAILED DESCRIPTION

Medical devices can be equipped with one or more sensing devices (e.g., sensors, electrodes) and programmed to sense physiological data such as electrocardiogram (ECG) data. To collect physiological data, one or more medical devices (e.g., implantable cardiac monitors/recorders, external cardiac monitors/recorders) can be implanted in or coupled to the patient such that the medical devices can sense the physiological data. Although such medical devices may attempt to continuously sense physiological data, to save power and memory the medical devices may not ultimately record and transmit all the sensed physiological data outside of the medical devices. Instead, the sensed physiological data may only be recorded and then transmitted in response to a potential cardiac event. For example, a patient could manually request that sensed physiological data be recorded (e.g., via a button, icon, etc., on a user device or the medical device itself). Such a request may occur if the patient believes that they are experiencing symptoms of a cardiac event. As another example, the medical devices themselves may be programmed to process and analyze the sensed physiological data to determine the occurrence of a potential cardiac event. If a potential cardiac event is determined, certain sensed physiological data can be recorded to longer-term memory (as opposed to a temporary buffer) and transmitted to another device. As yet another example, a separate device (e.g., sensor) external to the medical devices could determine the occurrence of a potential cardiac event (or another type of health-related event) and cause the physiological data to be recorded to longer-term memory and transmitted to another device.

Regardless of how recording to longer-term memory is initiated, the recorded physiological data associated with potential cardiac events may ultimately be assessed by a physician to determine whether and/or how treatment should be administered to the patient. However, the number and frequency of potential cardiac events can be overwhelming to process and assess. Further, the recorded physiological data associated with potential cardiac events may actually contain normal cardiac activity. This can occur when a patient—after feeling symptoms—initiates recordation of a potential cardiac event but the recorded physiological data shows that the patient's cardiac activity was normal at the time. This can also occur if a medical device initially incorrectly identifies normal cardiac activity as abnormal cardiac activity.

Certain instances of the present disclosure are accordingly directed to technology for assessing potential cardiac events before assessment by a human. In particular, certain instances of the present disclosure utilize computing devices and/or computing systems that can intercept, process, and assess physiological data sensed and recorded by a medical device before the physiological data is transmitted to a physician or technician for review.

### Cardiac Event Evaluation System

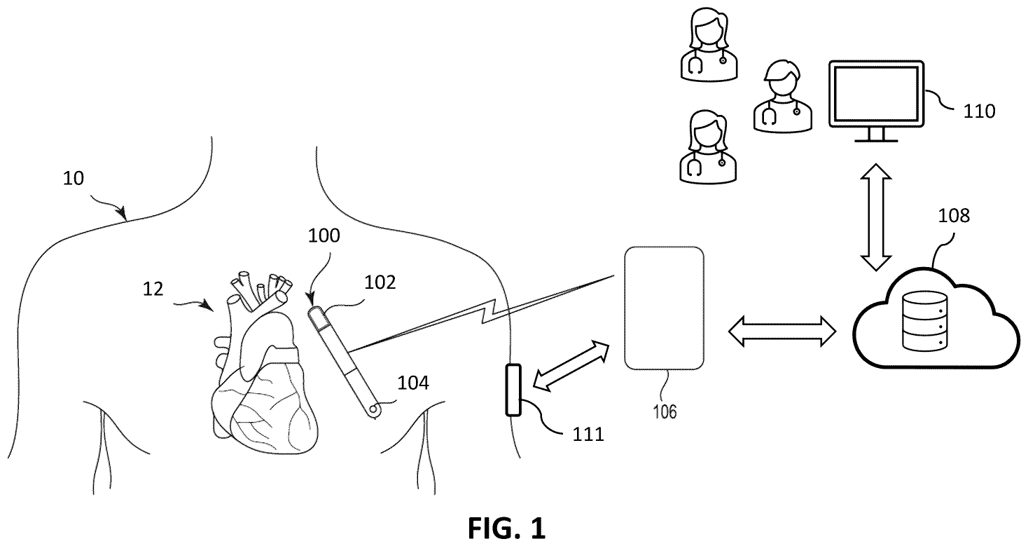

FIG. 1 is a schematic illustration of a medical device 100 within a cardiac event evaluation system. The medical device 100 can be implanted subcutaneously within an implantation location or pocket in the patient's chest or abdomen and may be configured to sense physiological signals associated with the patient's heart 12. The medical device 100 may be an implantable cardiac monitor (e.g., an implantable diagnostic monitor, an implantable loop recorder) configured and programmed to record physiological parameters such as, for example, one or more cardiac activation signals, heart sounds, blood pressure measurements, oxygen saturations. Although the medical device 100 of FIG. 1 is shown as an implantable cardiac monitor, the approaches described herein can be used in connection with other types of implantable medical devices such as a pulse generator (e.g., pacemaker, defibrillator). Further, although the medical device 100 of FIG. 1 is shown as a medical device that is implanted into the patient 10, the approaches described herein can be used in connection with an external medical device such as a monitoring device that is attached to a patient's skin or a mobile device that includes one or more sensors for detecting physiological data and/or that can process detected physiological data. Further yet, although the medical device 100 is described as a device that detects physiological data related to cardiac activity, the approaches described herein can apply to other types of physiological data which is used by physicians to determine whether treatment should be administered to the patient 10.

In the example of FIG. 1, the medical device 100 includes electrodes 102 and 104, which sense physiological signals (e.g., electrical signals) of the heart 12. The sensed physiological signals can be used to generate electrocardiogram (ECG) data such as ECG waveforms, which represent sensed cardiac electric activity over time. In certain instances, the medical device 100 initially and temporarily records sensed physiological data to buffer/cache memory and then—in certain circumstances—to longer-term memory. The sensed physiological data recorded to longer-term memory can be communicated (e.g., communicated wirelessly) to a receiver 106 (e.g., a mobile computing device, programmer, controller, patient monitoring system, mobile phone, smartphone, tablet computer, laptop computer, desktop computer external to the patient).

The receiver 106 can be communicatively coupled to the medical device 100 and positioned external to the patient's body. The receiver 106 can be a device that is capable of programming, controlling, monitoring, and/or otherwise communicating with the medical device 100. The receiver 106 can help facilitate communication from the medical device 100 to another device or system such as a computing system 108 (e.g., laptop computer, desktop computer, server). The receiver 106 and/or the computing system 108 can be communicatively coupled to another computing system 110 with a display on which users (e.g., patients, physicians, technicians) can view data sensed and recorded by the medical device 100.

The medical device 100 can be programmed to repeatedly delete a certain amount of historical sensed physiological data (e.g., from the buffer/cache memory) until the occurrence of a potential cardiac event. For example, the medical device 100 may continuously delete historical data (that is not associated with a potential cardiac event) after expiration of a rolling period of time (e.g., 10-60 seconds) to preserve memory capacity and power. However, once a potential cardiac event occurs, the medical device 100 can be programmed to begin storing the sensed physiological data until the potential cardiac event is determined to be complete or until a predetermined amount of time has elapsed. This may include storing physiological data that had not already been deleted as well as a certain amount of physiological data sensed after completion of the potential cardiac event (e.g., 20-60 seconds of sensed physiological data).

As noted above, in certain instances, a patient could request that sensed physiological data be recorded such as in response to a potential cardiac event (e.g., in the event the patient experiences symptoms such as an increased heart rate). As another example, the medical device 100 itself may be programmed to process and analyze the sensed physiological data to determine the occurrence of a potential cardiac event. As yet another example, a separate device (e.g., sensor 111) external to the medical devices could determine the occurrence of a potential cardiac event (or other health-related event) and cause the physiological data to be recorded by the medical device 100.

Potential cardiac events may ultimately be evaluated by a physician to determine whether and/or how treatment should be administered to the patient. However, to reduce expending resources on transmitting and evaluating normal cardiac activity, the cardiac event evaluation system can utilize approaches for assessing potential cardiac events before notifying or otherwise transmitting the underlying physiological data to physicians for evaluation.

### Assessing Potential Cardiac Events

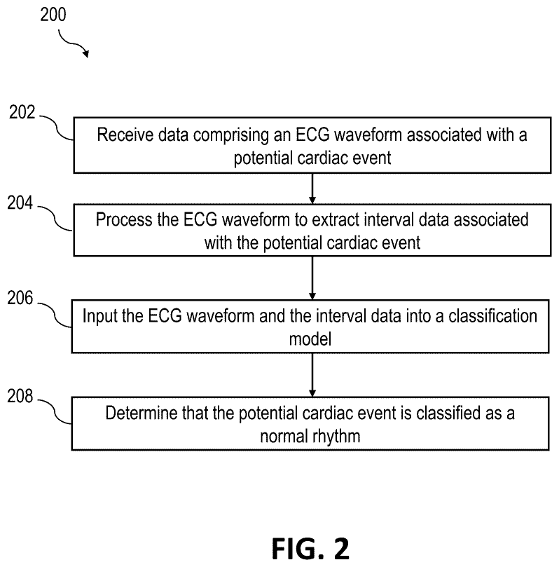

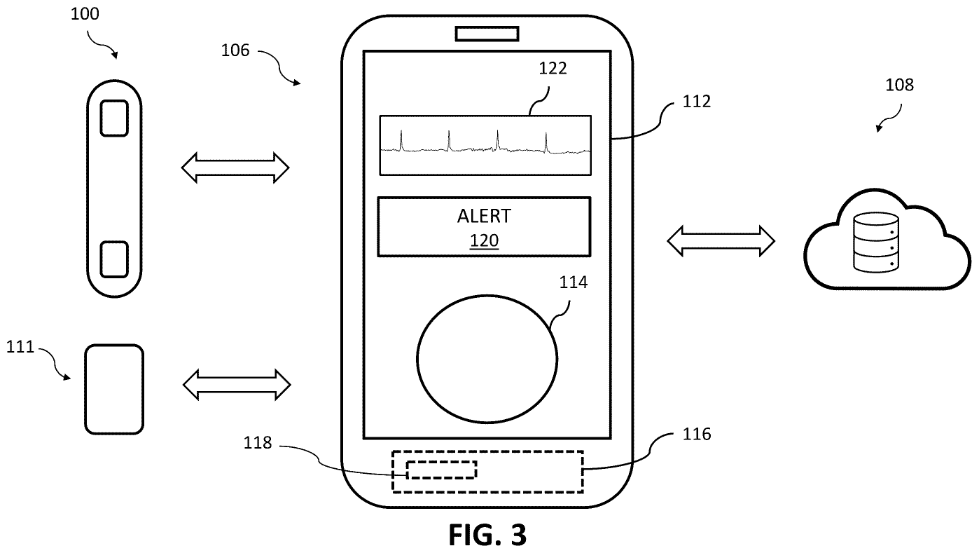

FIG. 2 outlines steps of a method 200 for assessing potential cardiac events before evaluation from a human. The method 200 outlined in FIG. 2 is described in connection with features of FIG. 3, which shows additional details of the receiver 106 initially shown in FIG. 1.

The method 200 includes receiving a package of data comprising an ECG waveform associated with a potential cardiac event (block 202 in FIG. 2). This package of data can be received by the receiver 106 or another device within the system of FIG. 3. The package of data can be generated by the medical device 100. For example, the medical device 100 can sense physiological data (including an ECG waveform), and the ECG waveform can be recorded in response to receiving input from a patient indicating that the patient is experiencing a potential cardiac event. The patient can provide the input via a user interface 112 (e.g., an interface shown on a display, screen, and the like) on the receiver 106. For example, the user interface 112 can include a button or icon 114 that the patient can select, press, contact, etc., to request that the medical device 100 record physiological data. In response to receiving the input/request from the patient, the receiver 106 can transmit a command to the medical device 100 to record the sensed physiological data (including an ECG waveform). The medical device 100 can then initiate recording of the sensed physiological data (e.g., an ECG waveform) to longer-term memory (e.g., memory other than buffer/cache memory). For example, the medical device 100 can be programmed to record a predetermined length of ECG waveform (e.g., a predetermined length of ECG waveform data occurring before and after the patient's input). The recorded ECG waveform can then be transmitted from the medical device 100 to another system and/or another device such as the receiver 106.

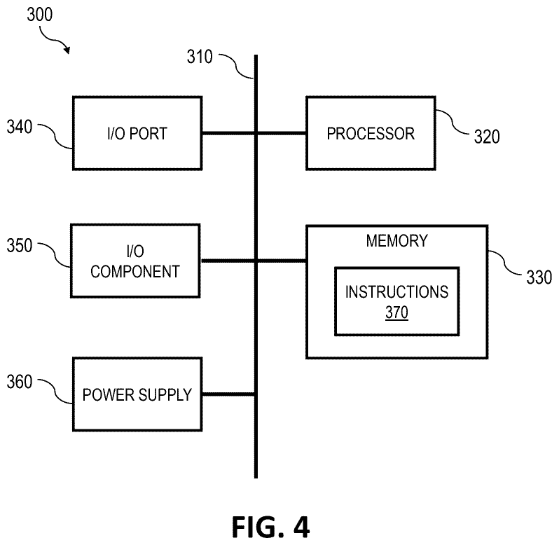

Referring back to FIG. 2, the method 200 can further include processing the ECG waveform to extract time interval data associated with the potential cardiac event (block 204 in FIG. 2). For example, the receiver 106 can be programmed to process the ECG waveform and generate time interval data, which describes features relating to intervals within the ECG waveform. The receiver 106 can include a computing device 116 (shown in more detail in FIG. 4 described below) that can be programmed to carry out the various functions described herein, including extracting interval data based on the ECG waveform. For example, the computing device 116 can be programmed to apply one or more algorithms to identify peaks within the ECG waveform and/or wavelet-based filtering. More specifically, a Pan-Tompkins algorithm can be used for QRS detection. Additionally or alternatively, a Multivalued Teager Energy Operator (MTEO) algorithm can identify peaks through filters sensitive to sharp spikes (e.g., spikes in ECG waveform observed during R-peaks).

Interval data extracted from (or otherwise based on the ECG waveform) can include data such as RR intervals, which represent the time elapsed between two successive R-waves of QRS portions of a beats within an ECG waveform. Put another way, RR intervals measure the time between two peaks of successive R-waves. Other examples of interval data based on the ECG waveform include a number of R peaks, mean amplitude of R peaks, standard deviation of amplitudes of R peaks, root mean squared (RMS) values of adjacent R peaks, number of R waves, number of Q waves, number of T waves, mean RR interval, mean QT interval, mean TQ interval, mean PR interval, QRS width, mean QRS interval, mean TQ interval, mean ST interval, mean corrected TQ intervals (TQc), mean corrected QT intervals (QTc), ratio of mean TQc and QTc, standard deviations of the various intervals and RMS of the various intervals.

Additionally or alternatively, the receiver 106 can be programmed to process the ECG waveform and generate nonlinear features. Example nonlinear features include approximate entropy (e.g., a measure used to quantify an amount of regularity and the unpredictability of fluctuations over time-series data), correlation dimensions (e.g., a measure of dimensionality of space occupied by a set of random points), and Lyapunov exponents (e.g., a quantity that characterizes the rate of separation of infinitesimally close trajectories).

Additionally or alternatively, the receiver 106 can be programmed to process the ECG waveform and generate logarithmic scattering coefficients or wavelet scattering coefficients. These coefficients can be obtained using wavelet transform (e.g., convolution), modulus (e.g., nonlinearity), and low-pass filtering (e.g., averaging) to generate time invariant signal coefficients.

After the ECG waveform has been processed, the ECG waveform and the interval data (and/or other types of data) can be inputted into a classification model (block 206 in FIG. 2). The computing device 116 can be programmed to operate the classification model 118 (represented in FIG. 3). In certain instances, the classification model 118 is a machine learning model trained to process an ECG waveform and data (such as interval data, nonlinear features, and/or scattering coefficients) to classify a potential cardiac event. In such instances, the machine learning model can include models that utilize approaches as boosted trees (e.g., an ensemble of boosted trees) or gradient boosting (e.g., gradient boosted decision trees).

The method 200 further includes determining, by the classification model 118, that the potential cardiac event is classified as a normal rhythm (block 208 in FIG. 2). In certain instances, the classification model 118 is trained to make a binary decision (e.g., the potential cardiac event is either a normal cardiac rhythm or an abnormal cardiac rhythm). More specifically, the classification model 118 can be trained to identify whether the ECG waveform contains or does not contain normal sinus rhythms based on the ECG waveform (and other data such as interval data associated with the ECG waveform). It has been found that, in certain instances, training and operating the classification model 118 to make a binary decision results in fewer false positive determinations of normal sinus rhythms.

If the classification model 118 identifies (or classifies) the potential cardiac event as a normal cardiac rhythm (e.g., normal sinus rhythm), the computing device 116 can be programmed to generate an alert. The alert can be displayed on the user interface 112 in a window 120 or tile and can include or be accompanied with a message such as “Normal Rhythm Detected” or something similar. The user interface 112 can also display one or more relevant portions of the ECG waveform 122 so that the patient can view the ECG waveform recorded in connection with the potential cardiac event.

In certain instances, when the classification model 118 identifies (or classifies) the potential cardiac event as a normal cardiac rhythm, the computing device 116 is programmed to prevent the patient's physician from being notified of the potential cardiac event. In this example, the receiver 106 (e.g., via its computing device 116) is able to intercept potential cardiac events, make an initial assessment or classification, and prevent sending the patient's physician alerts about potential cardiac events that are determined to contain normal cardiac activity. This approach can reduce the number of events physicians are alerted to or that are otherwise manually reviewed by physicians.

However, in other instances, the patient's physician may still want to review all patient-initiated potential cardiac events regardless of the initial assessment. In these instances, the classification model 118 can still determine whether the potential cardiac event contains normal cardiac activity, but the computing device 116 can generate metadata (e.g., a data flag or indicator) that indicates that the potential cardiac event was determined to include normal cardiac activity. The metadata can be transmitted to the physician (along with the ECG waveform and other data) to indicate which events were assessed as containing normal cardiac rhythms.

If the classification model 118 does not identify a normal cardiac rhythm with the potential cardiac event, the computing device 116 can be programmed to forward the potential cardiac event (and the ECG waveform) to the patient's physician. Further, the computing device 116 can generate an alert. The alert can be displayed on the user interface 112 in the window 120 or tile and can include or be accompanied with a message such as “Other rhythm. Ongoing review. Clinic will reach out if necessary.”

A physician can access patients' potential cardiac events (and the underlying data) via the computing system 110 and view the information on the display. For example, after the sensed physiological data and metadata associated with potential cardiac events is transmitted from the medical device 100, the computing system 108 such as one or more servers can provide selective access to such data via the computing system 110. In certain instances, the computing system 108 hosts a software-as-a-service (SaaS) platform (e.g., an online platform), which can be accessed by approved users via a web browser on the computing system 110.

In addition to ECG waveform and interval data, other types of data can be used as an input to classify or otherwise estimate normal cardiac activity versus abnormal cardiac activity. For example, the medical device 100 could include an acceleration sensor (e.g., an accelerometer) or the sensor 111 could be an acceleration sensor, and the acceleration sensor(s) can be used to measure the patient's activity (e.g., exercise, noise) or lack of activity. The patient's activity can be an input to classify or otherwise estimate normal cardiac activity versus abnormal cardiac activity. The patient's activity can also be determined by a respiratory sensor within the medical device 100 and/or as the sensor 111. The patient's activity can also be determined by a chemical sensor within the medical device 100 and/or as the sensor 111. The chemical sensor could be configured to collect data such as lactate/enzyme levels in sweat, oxygenation levels in the blood, and the like.

Other cardiac-related data could be used as an input to classify or otherwise estimate normal cardiac activity versus abnormal cardiac activity. For example, heart sounds measured by the medical device 100 and/or the sensor 111 could be used to confirm classification of cardiac activity. As another example, a separate device could sense ECG waveforms, and the additional ECG waveform could be used to confirm classification of cardiac activity.

The additional sensor data listed in the two preceding paragraphs could be an input to the classification model 118 or could be used as a separate confirmation check by the computing device 116 before the ECG waveform is transmitted to a physician for assessment.

In addition to ECG waveform and interval data, other types of data can be used to determine whether to identify a potential cardiac event, generate an alert, and/or notify a physician. For example, sensor data (e.g., data from the sensor 111 and/or other sensors) can be transmitted to the computing device 116. This sensor data can include data indicating other conditions the patient is experiencing such as data related to heart failure, chronic kidney disease, chronic obstructive pulmonary disease, etc.

The computing device 116 can be programmed to transmit the ECG waveform (and other physiological data) to a physician in the event the computing device 116 determines that the sensor data indicates that one or more of the patient's conditions are worsening (e.g., based on crossing a threshold).

### Computing Devices and Systems

FIG. 4 is a block diagram depicting an illustrative computing device 300 such as a mobile computing device, in accordance with instances of the disclosure. Each of the various components shown and described in the Figures can contain their own dedicated set of computing device components shown in FIG. 4 and described below. For example, the receivers, servers, and remote computers can each include their own set of components shown in FIG. 4 and described below.

In instances, the computing device 300 includes a bus 310 that, directly and/or indirectly, couples one or more of the following devices: a processor 320, a memory 330, an input/output (I/O) port 340, an I/O component 350, and a power supply 360. Any number of additional components, different components, and/or combinations of components may also be included in the computing device 300.

The bus 310 represents what may be one or more busses (such as, for example, an address bus, data bus, or combination thereof). Similarly, in instances, the computing device 300 may include a number of processors 320, a number of memory components 330, a number of I/O ports 340, a number of I/O components 350, and/or a number of power supplies 360. Additionally, any number of these components, or combinations thereof, may be distributed and/or duplicated across a number of computing devices.

In instances, the memory 330 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 330 stores computer-executable instructions 370 for causing the processor 320 to implement aspects of instances of components discussed herein and/or to perform aspects of instances of methods and procedures discussed herein. The memory 330 can comprise a non-transitory computer readable medium storing the computer-executable instructions 370.

The computer-executable instructions 370 may include, for example, computer code, machine-useable instructions, and the like such as, for example, program components capable of being executed by one or more processors 320 (e.g., microprocessors) associated with the computing device 300. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 370 may be configured to be executed by the processor 320 and, upon execution, to cause the processor 320 to perform certain processes. In certain instances, the processor 320, memory 330, and instructions 370 are part of a controller such as an application specific integrated circuit (ASIC), field-programmable gate array (FPGA), and/or the like. Such devices can be used to carry out the functions and steps described herein.

The I/O component 350 may include a presentation component configured to present information to a user such as, for example, a display device, a speaker, and/or the like, and/or an input component such as, for example, a microphone, a joystick, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
a mobile computing device comprising memory and one or more processors, wherein the memory stores instructions that, when executed, cause the mobile computing device to:
process electrocardiogram (ECG) waveform associated with a potential cardiac event to extract interval data associated with the potential cardiac event,
input the ECG waveform and the interval data into a trained machine learning model, and
determine, by the trained machine learning model, that the potential cardiac event comprises a normal rhythm.

2. The system of claim 1, wherein the instructions, when executed, further cause the mobile computing device to:
generate an alert in response to the determining that the potential cardiac event contains the normal cardiac rhythm.

3. The system of claim 2, wherein the instructions, when executed, further cause the mobile computing device to:
display the alert and the ECG waveform on a display of the mobile computing device.

4. The system of claim 1, wherein the interval data comprises data relating to peaks of R waves.

5. The system of claim 4, wherein the data relating to peaks of R waves includes a time interval between successive R waves.

6. The system of claim 1, wherein the instructions, when executed, further cause the mobile computing device to:
process the ECG waveform to generate non-linear features, and
input the non-linear features into the trained machine learning model.

7. The system of claim 1, wherein the instructions, when executed, further cause the mobile computing device to:
prevent the potential cardiac event from being forwarded to a physician.

8. The system of claim 1, wherein the trained machine learning model comprises classification model.

9. The system of claim 1, wherein the trained machine learning model comprises an ensemble of boosted trees.

10. The system of claim 1, wherein the instructions, when executed, further cause the mobile computing device to:
determine, based on sensor data other than the ECG waveform, that a condition of a patient is worsening, and generate an alert in response to the condition worsening.

11. The system of claim 1, wherein the instructions, when executed, further cause the mobile computing device to:
transmit a command to a medical device to record the ECG waveform, in response to input from the patient.

12. The system of claim 11, wherein the mobile computing device includes a display configured to display a user interface, wherein the input is a selection of an icon on the user interface.

13. The system of claim 11, further comprising:
the medical device communicatively coupled to the mobile computing device, wherein the medical device is programmed to record the ECG waveform in response to the medical device receiving the command.

14. The system of claim 1, wherein the mobile computing device is programmed to determine, via the trained machine learning model, that the potential cardiac event is only either an abnormal cardiac event or a normal cardiac event.

15. A method comprising:
receiving a package of data comprising an electrocardiogram (ECG) waveform associated with a potential cardiac event;
processing the ECG waveform to extract interval data associated with the potential cardiac event;
inputting the ECG waveform and the interval data into a trained machine learning model; and
determining, by the trained machine learning model, that the potential cardiac event is either a normal cardiac event or an abnormal cardiac event.

16. The method of claim 15, further comprising:
generating an alert in response to the determining that the potential cardiac event is the normal cardiac event; and
displaying the alert and the ECG waveform on a display of a mobile computing device.

17. The method of claim 15, wherein the interval data comprises data relating to peaks of R waves.

18. The method of claim 15, further comprising:
preventing the potential cardiac event from being forwarded to a physician.

19. The method of claim 15, further comprising:
receiving input from a patient indicating that the patient is experiencing the potential cardiac event; and
transmitting a command to a medical device to record the ECG waveform, in response to the input from the patient.

20. The method of claim 19, further comprising:
recording the ECG waveform, by the medical device, in response to the medical device receiving the command.
